(19) United States
(12) Patent Application Publication
(10) Pub. No.: US 2016/0220122 A1
(43) Pub. Date: Aug. 4, 2016

(54) PHYSIOLOGICAL CHARACTERISTICS DETERMINATOR

(71) Applicants: Michael Edward Luna, Broadmoor Village, CA (US); Thomas Alan Donaldson, Drews Cottage (GB); John M. Stivoric, Pittsburgh, PA (US); Sidney Primas, Palo Alto, CA (US)

(72) Inventors: Michael Edward Luna, Broadmoor Village, CA (US); Thomas Alan Donaldson, Drews Cottage (GB); John M. Stivoric, Pittsburgh, PA (US); Sidney Primas, Palo Alto, CA (US)

(73) Assignee: AliphCom, San Francisco, CA (US)

(21) Appl. No.: 15/006,096

(22) Filed: Jan. 25, 2016

Related U.S. Application Data
(60) Provisional application No. 62/107,411, filed on Jan. 25, 2015.

(51) Int. Cl. A61B 5/0205; A61B 5/11; A61B 5/00

(52) U.S. Cl. CPC A61B 5/0205; A61B 5/7275; A61B 5/11; A61B 5/6801; A61B 2562/0219; A61B 2560/0223

(57) ABSTRACT

One or more wearable devices may measure real-time blood pressure in a body using signals from multiple sensors including but not limited to a multi-axis accelerometer, a bioimpedance (BI) sensor, a capacitive touch sensor, an electrocardiography sensor (ECG), a ballistocardiograph sensor (BCG), a photoplethysmogram (PPG), a pulse oximetery sensor, and a phonocardiograph sensor (PCG), for example. Accelerometry data (e.g., from a multi-axis accelerometer or BCG sensor) may be used to derive effects of acceleration (e.g., gravity) on changes in blood pressure (e.g., due to changes in blood volume as measured using BI signals). The accelerometry data may be used to determine a baseline value for BI voltage signals that are indicative of diastolic and systolic blood pressure (e.g., in mmHg). Combinations of methods, such as BCG, ECG, PPG, blood pressure Pulse Wave and others may be used to determine pulse transit time (PTT), pulse arrival time (PAT), and pre-ejection period (PET). The wearable devices may be born on one or more body parts, such as the wrist, arm, leg, ankle, neck, chest, thorax, head, and ear.

### CROSS-RELATED APPLICATIONS

This application claims benefit and right of priority under 35 U.S.C. §119(e) to the following U.S. Provisional Patent Application: U.S. Provisional Patent Application No. 62/107,411, filed on Jan. 25, 2015, and titled “PHYSIOLOGICAL CHARACTERISTICS DETERMINATOR”, which is herein incorporated by reference in its entirety for all purposes. This application is related to the following application: U.S. patent application Ser. No. 14/209,690, filed on Mar. 13, 2014, and titled “EAR-RELATED DEVICES IMPLEMENTING SENSORS TO ACQUIRE PHYSIOLOGICAL CHARACTERISTICS”; which is herein incorporated by reference in its entirety for all purposes.

### FIELD

Embodiments of the present application relate generally to electrical and electronic hardware, computer software, sensors, biometric sensors, bioimpedance sensors, wired and wireless communications, wireless devices, wearable devices, medical devices, and consumer electronic devices.

### BACKGROUND

Conventional blood pressure measurements may require clinical instruments, such as a blood pressure cuff (e.g., a sphygmomanometer) to take a blood pressure reading for systolic and diastolic pressure (e.g., in mmHg). Subsequently, the blood pressure reading may be used as a baseline with other biometric data, such as bioimpedance data, to derive a value of blood pressure from the bioimpedance data. However, obtaining the baseline blood pressure data requires cooperation and availability of the person who is the subject of the blood pressure readings. Further, a person may typically be required to sit and be still, and to rest an arm being measured on a surface such as a table or an arm of a chair. Additionally, the use of the blood pressure readings as a baseline for calculating blood pressure using the biometric data may lead to inaccurate blood pressure determinations due to changes in actual blood pressure caused by activity such as exercise, sleep, rest, arousal, stress, and illness, just to name a few.

Accordingly, there is a need for systems, apparatus and methods to determine clinically accurate blood pressure in-situ, in real-time, from multiple sensor inputs.

### BRIEF DESCRIPTION OF THE DRAWINGS

Various embodiments or examples (“examples”) are disclosed in the following detailed description and the accompanying drawings:

FIG. 1 depicts an example of a waveform indicative of blood pressure;

FIG. 2 depicts an example of multiple inputs of which one or more may be used for determining blood pressure using signals and/or data associated with one or more of the multiple inputs;

FIG. 3 depicts one example of a block diagram for a system;

FIG. 4 depicts one example of a bioimpedance waveform;

FIG. 5 depicts an example of a computing resource and a data resource; and

FIG. 6 depicts an example of a portion of a wearable device;

FIG. 7 depicts one example of a block diagram for a calibration system;

FIG. 8 depicts another example of block diagram for a calibration system;

FIG. 9 depicts examples of waveforms for sensor signals;

FIG. 10 depicts examples of body motion and sensor signals generated by the body motion that may be used for calibration;

FIG. 11 depicts examples of signals generated individually or in subsets of two or more signals;

FIG. 12 depicts an example of a pressure calculator configured to determine blood pressure;

FIG. 13 depicts an example of a correlator engine configured to access a database; and

FIG. 14 depicts an example of a computing platform that may be disposed in a wearable device.

Although the above-described drawings depict various examples of the invention, the invention is not limited by the depicted examples. It is to be understood that, in the drawings, like reference numerals designate like structural elements. Also, it is understood that the drawings are not necessarily to scale.

### DETAILED DESCRIPTION

Various embodiments or examples may be implemented in numerous ways, including but not limited to implementation as a system, a process, a method, an apparatus, a user interface, or a series of executable program instructions included in a non-transitory computer readable medium. Such as a non-transitory computer readable medium or a computer network where the program instructions are sent over optical, electronic, or wireless communication links and stored or otherwise fixed in a non-transitory computer readable medium. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

Although the foregoing examples have been described in some detail for purposes of clarity of understanding, the above-described conceptual techniques are not limited to the details provided. There are many alternative ways of implementing the above-described conceptual techniques. The disclosed examples are illustrative and not restrictive.

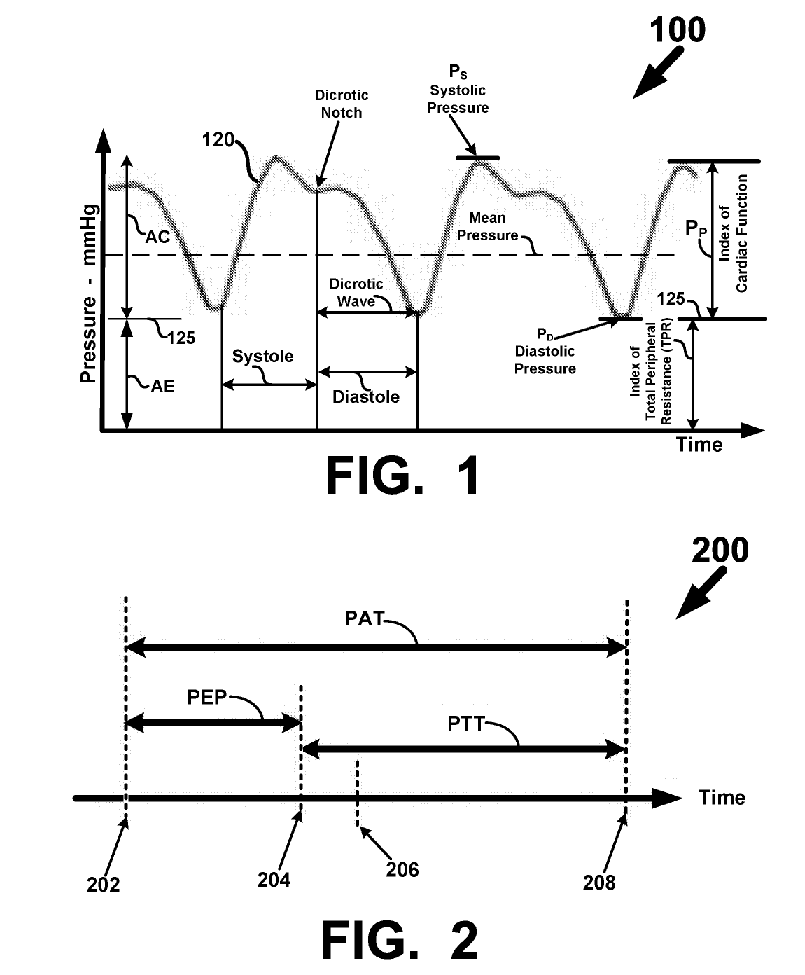

FIG. 1 depicts an example 100 of a waveform 120 indicative of blood pressure. A y-axis indicates pressure in mmHg and an x-axis indicates time. The waveform 120 may indicative of a blood pressure waveform in an artery (e.g., a radial artery of a wrist). The waveform 120 may be a bioimpedance waveform, for example. One or more sensors that may be used to generate signals indicative of blood pressure (e.g., changes in blood volume as blood flows through the artery) may include signal artifacts caused by motion of the body the sensors are coupled to, such as arm motion for a sensor disposed on a wrist, limb motion for a sensor disposed on one of the appendages of the body, head or other body motion for a sensor disposed on an ear, the neck, thorax, or the head, for example. According to some examples, a motion detector (e.g., an accelerometer and/or a multi-axis accelerometer) may generate accelerometry data representative of body motion that produces at least a portion of the signal artifacts in waveform 120. Accelerometry data may be indicative of effects of gravity (e.g., as measured in G′s) on blood pressure.

In FIG. 1, accelerometry contributions to the waveform 120 may be factored out to determine baseline values (e.g., in voltage, current, or data) indicative of diastolic pressure P_{D} (e.g., a voltage minimum) and systolic pressure P_{S} (e.g., a voltage maximum). In example 100, a region below line 125 may be indicative of an index of total peripheral resistance (TPR) and a region above line 125 may be indicative of an index of cardiac function denoted by an arrow for pulse pressure P_{P}. Data and/or signals (e.g., from sensors) from a characterization process may be used to extract accelerometry (AE) effects from the signal indicative of blood pressure such that the effects of accelerometry opposing blood flow in systemic circulation through the artery may be reduced or eliminated from the signal indicative of blood pressure. The index of cardiac function may be derived by an automatic calibration (AC) of the signal indicative of blood pressure to provide waveform 120 that more accurately indicates values for P_{D}, P_{S}, and P_{P}, for example.

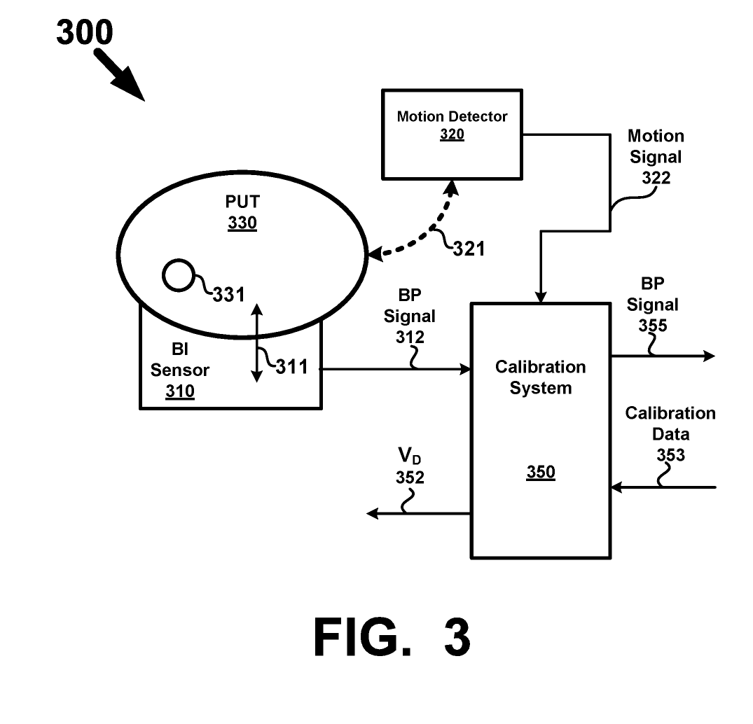

FIG. 2 depicts an example 200 of multiple inputs of which one or more of the multiple inputs may be used in determining blood pressure using signals and/or data associated with one or more of the multiple inputs. Data and/or signals from a body donned wearable device or from an external device may be used to determine one or more of the multiple inputs using one or more of: pulse transit time (PTT); pulse arrival time (PAT), and pre-ejection period (PEP), for example. The multiple inputs may constitute data and/or signals from a data store (e.g., a network, a data warehouse, Cloud storage, a database), and/or sensors used for accelerometry (e.g., a multi-axis accelerometer and/or a gyroscope), bioimpedance (BI), capacitive touch, an altimeter, electrocardiography (ECG), ballistocardiography (BCG), photoplethysmography (PPG), pulse oximetery, and phonocardiography (PCG), for example.

In FIG. 2, line 202 may be indicative of an ECG wave, such as a Q-wave, for example. Line 204 may be indicative of opening of the aortic valve, line 206 may be associated with maximum blood acceleration (e.g., a BCG J-wave). Line 208 may be indicative of a blood pulse wave arriving (e.g., a maximum point on a PPG slope) at a site in the body (e.g., at the wrist and/or at the ear).

FIG. 3 depicts one example 300 of a block diagram for a system. In FIG. 3, a body portion under test (PUT) 330 (e.g., a wrist of an arm and/or an ear) may include a structure from which a bioimpedance signal may be sensed using a bioimpedance (BI) sensor 310 coupled 311 with the PUT 330. Bioimpedance sensor 310 may include a plurality of electrically conductive structures, such as electrodes (not shown), that may contact a surface (e.g., of the skin) of a portion of the PUT 330, such as an area of skin proximate to an artery 331 (e.g., a radial artery in a wrist) in an interior portion of the PUT 330. In example 300, a motion detector 320 (e.g., a multi-axis accelerometer) may be coupled 321 with PUT 330 via a structure such as a device, a strap ban, a wrist band, or a watch band (wearable device hereafter), that includes the motion detector 320, for example. In other examples, motion detector 320 may be external to the wearable device, but may generate motion signals that are indicative of motion (e.g., accelerometry) imparted to the PUT 330. For example, motion detector 320 may be included in an external computing device such as a smartphone, tablet, wireless computing device, a bicycle, or an automobile, for example. Motion detector 320 may generate a motion signal 322 indicative of motion imparted to PUT 330 and/or a body the PUT 330 may be connected with, for example.

A calibration system 350 may receive the BI signal 312, the motion signal 322 and/or data representing those signals (e.g., signals converted from an analog domain format to a digital domain format). Motion signal 322 and/or BI signal 312 may be signals represented as a voltage, a current or a digital value (e.g., via conversion from analog to digital using an ADC). Calibration system 350 may communicate voltage data V_{D} 352 to one or more resources that may be internal to calibration system 350, external to calibration system 350 or both. Calibration system 350 may receive calibration data 353 determined by one or more resources that may be internal to calibration system 350, external to calibration system 350 or both. The calibration data 353 may be determined at least in part by the voltage data V_{D} 352 that was communicated by the calibration system 350. Calibration system 350 may use the calibration data 353 as a calibration factor. The calibration data 353 may be used in computations operative to remove motion related signal components from the BI signal to arrive at a blood pressure signal 355 (e.g., a voltage or data) indicative of the blood pressure in the PUT 330 (e.g., in mmHg).

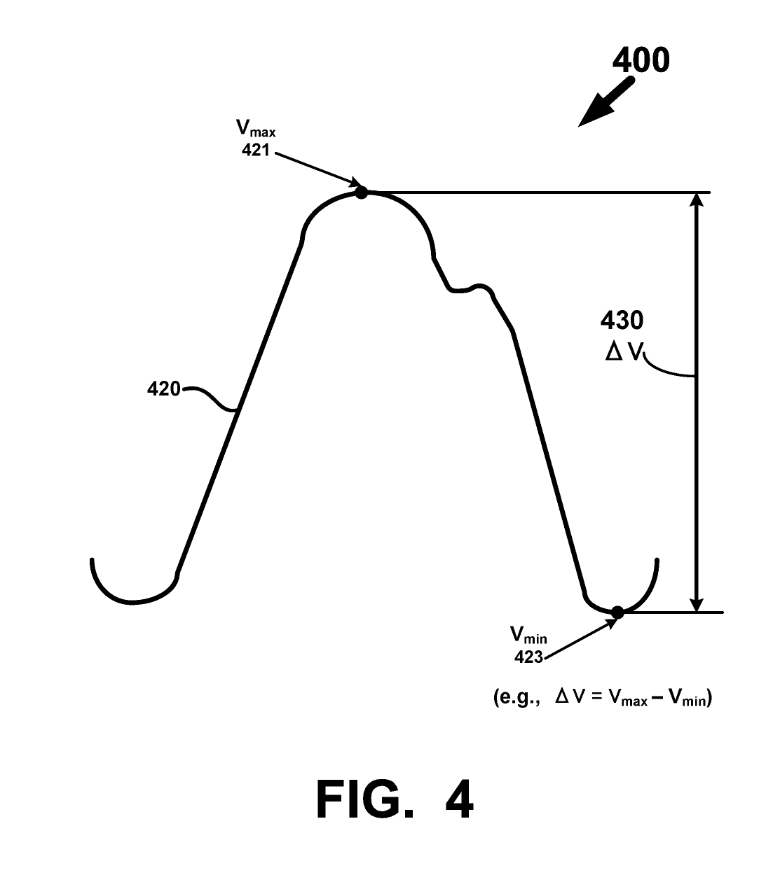

FIG. 4 depicts one example 400 of a bioimpedance waveform. Bioimpedance waveform 420 may include a voltage minimum Vmin 423 and a voltage maximum Vmax 421. A difference between Vmax 421 and Vmin 423 may be represented by ΔV 430 (e.g., ΔV 430=Vmax 421−Vmin 423). Calibration system 350 of FIG. 3 may receive ΔV 430 as BI signal 312. Bioimpedance waveform 420 may include contributions to changes in bioimpedance due to accelerometry. For example, motion of an arm up or down may cause changes in blood pressure that may manifest as changes in the bioimpedance signal. As another example, activity such as running, exercise, stress, resting, sleeping, or other activities of a user may have accelerometry associated with them, such as a higher accelerometry for running and a lower accelerometry for sleeping. Accordingly, ΔV 430 may be a measure of the bioimpedance signal that includes motion induced blood pressure artifacts that may be determined from motion signal 322 (see FIG. 3). For example, absent motion, the peak-to-peak value for ΔV 430 may be less than depicted in FIG. 4. However, in the presence of motion, blood pressure may be determined by factoring out the motion induced blood pressure artifacts, such that, actual blood pressure may be represented by BP signal 355 (see FIG. 3).

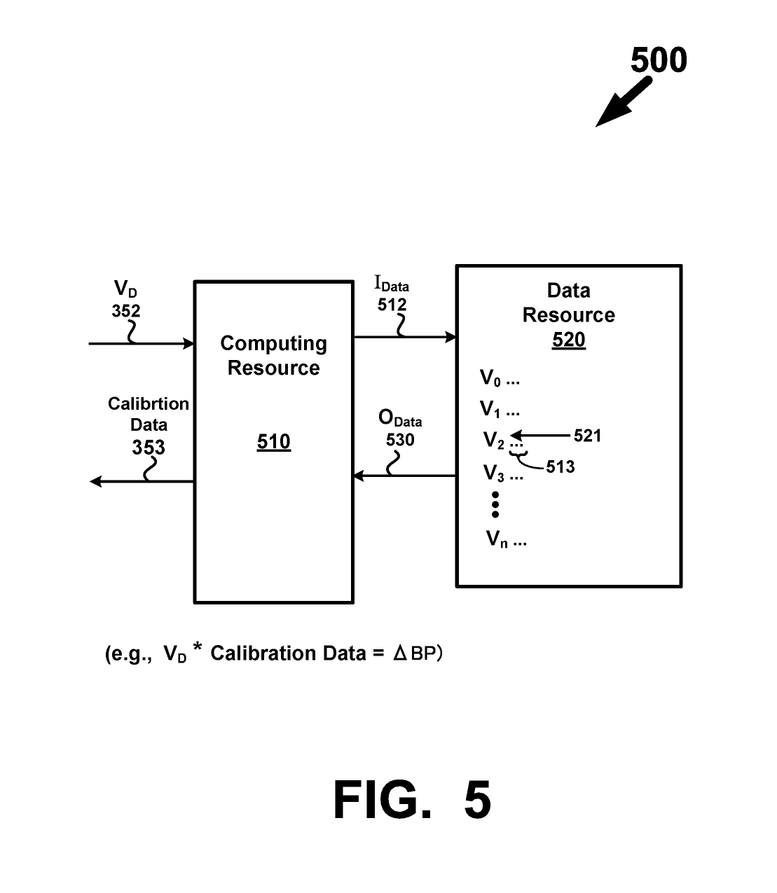

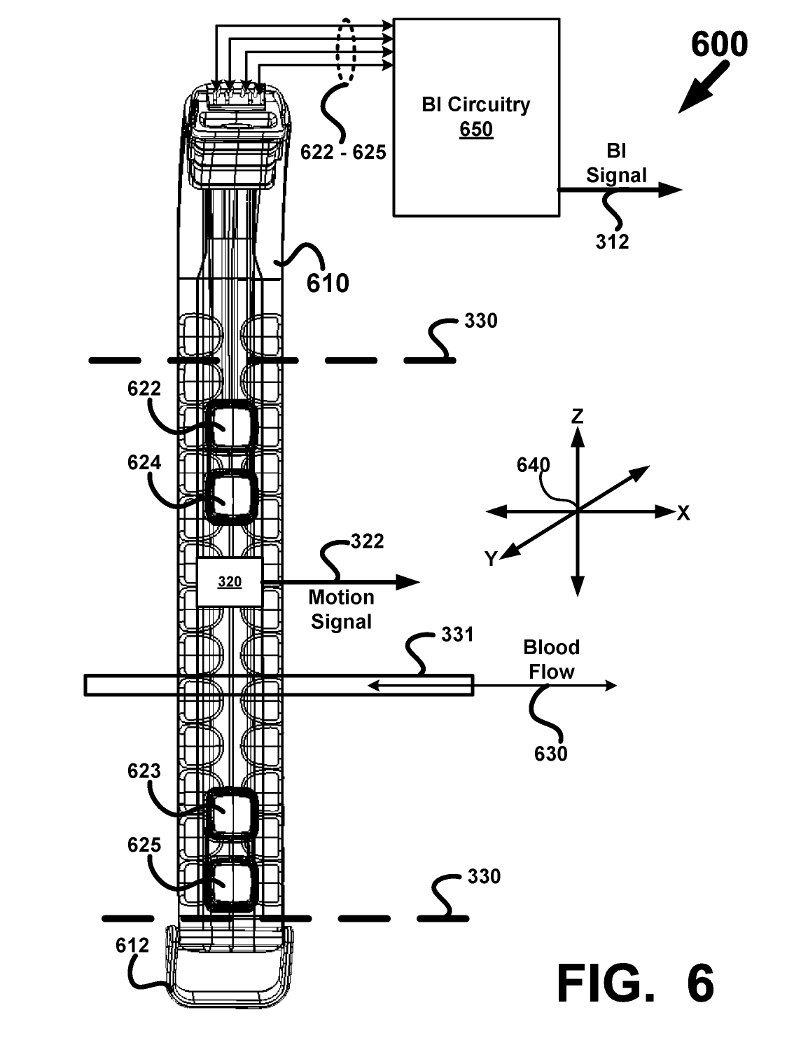

FIG. 5 depicts an example of a computing resource and a data resource. Computing resource 510 (e.g., a server, a microprocessor, a DSP, a controller) may receive voltage data V_{D} 352 (see FIG. 3). Computing resource 510 may communicate data representing the voltage data V_{D} 352 as input data I_{D} 512 to a data resource 520. Input data I_{D} 512 may be formatted (e.g., using computing resource 510) into an input vector format for a look-up-table (LUT) or other form of data structure (e.g., a data packet) in data resource 520. As one example, data resource 520 may include entries for data representing multiple voltage values denoted as V₀-Vₙ. Input data I_{D} 512 may be a match or an approximate match for entry V₂ 521. For example, Input data I_{D} 512 may be data representing a voltage value in voltage data V_{D} 352 (e.g., ΔV 430 in FIG. 4). An approximate match may include input data I_{D} 512 being closest in value to entry V₂ 521 (e.g., by +/−5% or less) than to values for entries V₁ and V₃, for example. Entries V₀-Vₙ may have a single value associated with them that may be used to match or closely match a corresponding value in the input data I_{D} 512, for example. Entries V₀-Vₙ may have a multiple values associated with them, denoted by 523, and the multiple values may be used to match or closely match corresponding multiple values in the input data I_{D} 512, for example. As one example, data representing multiple values in V₀-Vₙ may include but is not limited to data representing voltage (e.g., ΔV 430 in FIG. 4), data representing an age of a user, data representing a weight of a user, data representing a gender of a user, data representing an ethnicity of a user, data representing a race of a user, data representing demographic information of a user, data representing a larger pool or population of people, data representing a sub-pool or sub-population of people, anonymized data on a pool/population or sub-pool/sub-population of people, etc., just to name a few.

Data resource 520 may output data Odata 530 that may be received by computing resource 510. Computing resource may output data representing the output data Odata 530 as calibration data 353. Calibration system 350 of FIG. 3 may receive the calibration data 353 and may use the calibration data 353 to generate the BP signal 335. As one example, calibration data 353 may constitute data representing a calibration coefficient. Further to the example, the voltage value in voltage data V_{D} 352 may be 0.1V and data representing the 0.1V may be received as input data I_{D} 512 by data resource 520 may be returned as output data Odata 530, data representing a calibration coefficient of 5 (e.g., calibration data 353=5). Calibration system 350 may perform an operation (e.g., a mathematical operation) on voltage data V_{D} 352, such as multiplying voltage data V_{D} 352 by the calibration coefficient of 5 (e.g., 0.1×5=0.5). The resulting value may be indicative of the change in blood pressure in mmHg, such as a change in blood pressure of 0.5 mmHg, for example.

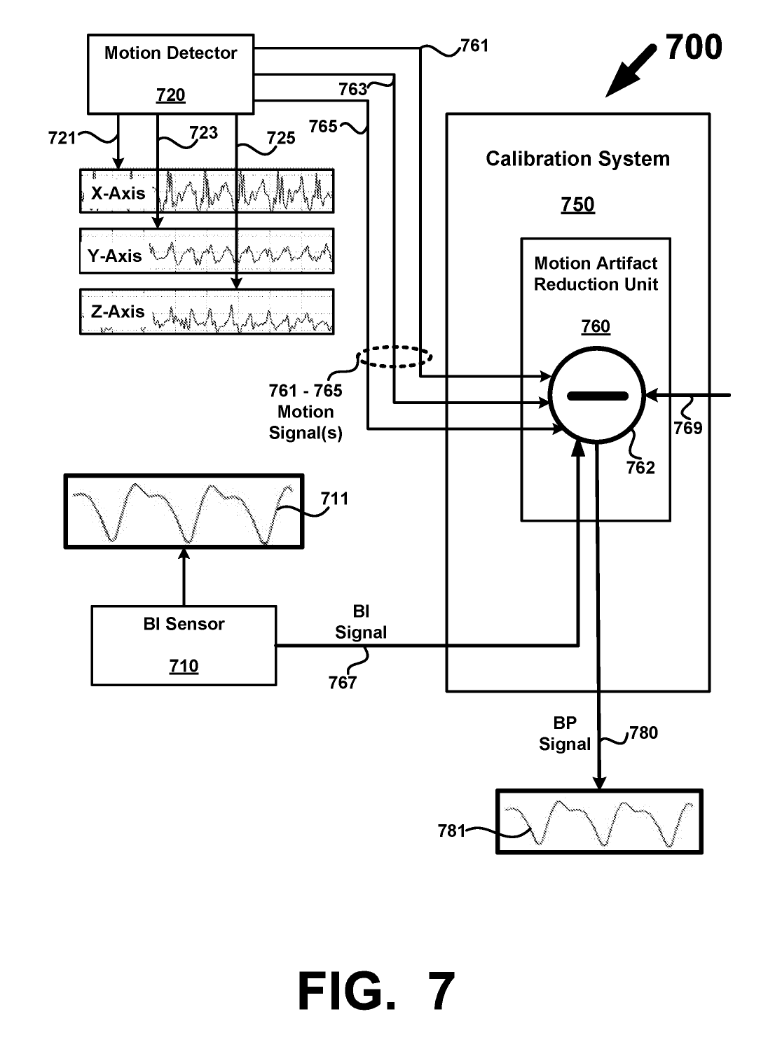

FIG. 6 depicts an example 600 of a portion of a wearable device. In FIG. 6, a portion 610 (e.g., a strap band) of a wearable device configured to be donned on a portion of the body of a user (e.g., PUT 330 in FIG. 3), may include electrodes 622, 624, 623 and 625 connected to portion 610. Electrically conductive traces may be routed from electrodes 622-625 and electrically coupled with bioimpedance circuitry (BI) 650. Bioimpedance circuitry 650 may include circuitry to drive a signal on one or more of the electrodes (e.g., apply signals to electrodes 622 and 625) and may include circuitry to receive bioimpedance signals from one or more other electrodes (e.g., receive signals from electrodes 624 and 623). Portion 610 or some other portion of the wearable device may include motion detector 320 (not shown). Motion detector 320 may generate one or more motion signals 322 indicative of acceleration relative to one or more motion axes (e.g., X, Y, Z axes of 640). Motion detector 320 may include one or more types of motion detectors including but not limited to one or more accelerometers, gyroscopes, and multi-axis accelerometers, for example.

Portion 610 may include a fastener 612 or other structure configured to mount or otherwise couple the wearable device to a portion of a body. Fastener 612 may couple with another fastener (not shown) to mount and/or adjust fit of the wearable device to the body. The wearable device may be configured, when donned, to position the electrodes 622-625 on portion 610 relative to a body structure to be sensed by the electrodes 622-625, such as artery 331. Bioimpedance signals received by the receiving electrodes (e.g., 623 and 624) may be indicative of changes in blood flow characteristic (e.g., blood pressure, blood volume) of blood flowing 630 through the artery 331, for example.

FIG. 7 depicts one example of a block diagram for a calibration system. Motion detector 720 may output one or more motion signals 761-765 related to motion signals for one or more axes 721-725, such as one or more of an X-axis, a Y-axis, or a Z-axis, for example. For example, BI sensor 710 may output a BI signal 767 representative of a BI waveform 711. Calibration system 750 may include a motion artifact reduction unit 760 being configured to receive signals or data representative of signals for BI signal 767, motion signals 761-765, and calibration data 769. Motion artifact reduction unit 760 may perform one or more operations 762 using the received data, such as subtracting out motion related components of the BI signal 767 that are due to one or more of the motion signals 761-765 to generate a blood pressure BP signal 780 having a waveform 781 indicative of blood pressure minus artifacts caused by accelerometry (e.g., motion of the body). Although FIG. 7 depicts a subtraction operation 762, the motion artifact reduction unit 760 may perform other operations on the data and/or signals received and the operation that may be performed are not limited to the subtraction example depicted. Operation 762 may include performing additional operations on a result of the operation using the calibration data 769. For example, the additional operations may include but are not limited to multiplication, addition, subtraction, division, interpolation, cubic spline interpolation, curve fitting, averaging, linear regression, or some combination of the foregoing.

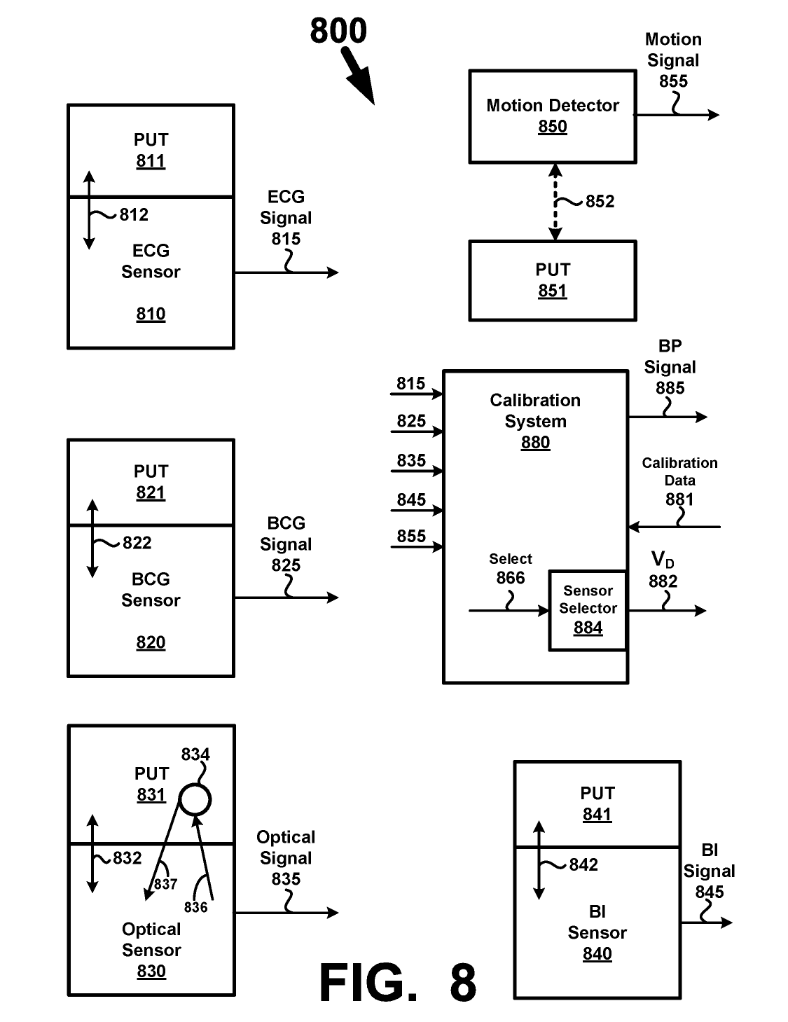

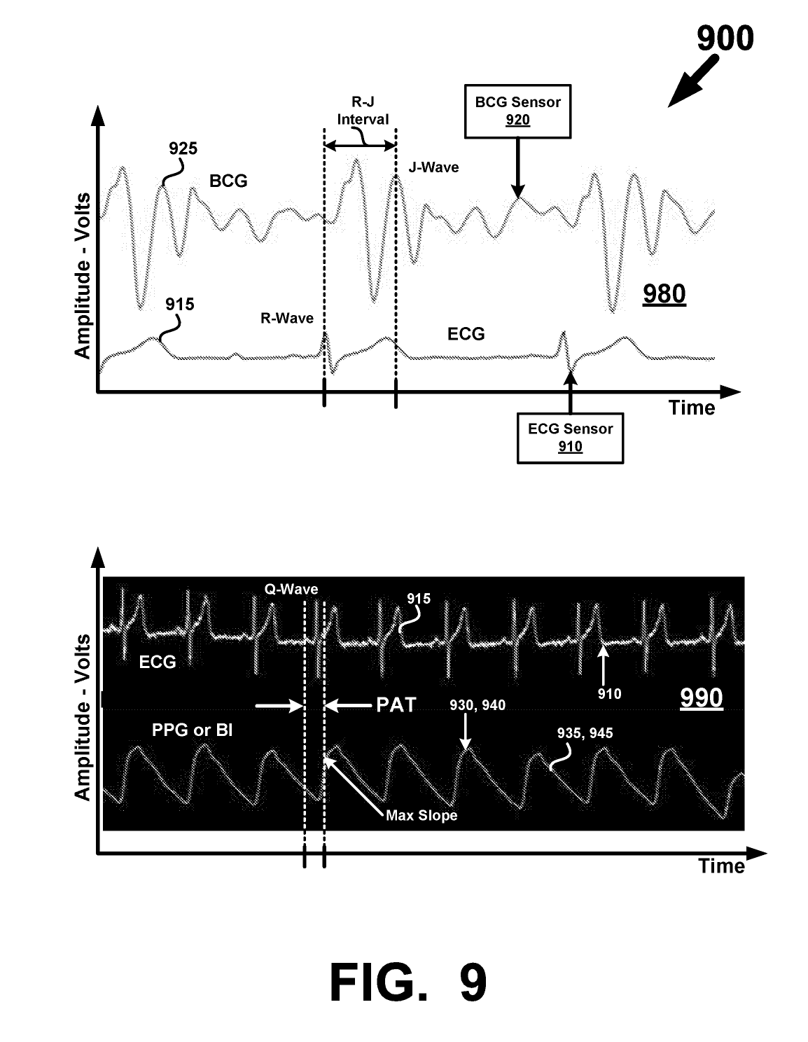

FIG. 8 depicts another example 800 of block diagram for a calibration system. In FIG. 8, a calibration system 880 may be coupled with signals from multiple sensor systems configured to detect signals associated with biometric data sensed from different portions of a body. An electrocardiogram sensor (ECG) 810 may be coupled 812 with a body portion under test (PUT) 811 and may generate an ECG signal 815. A ballistocardiogram sensor (BCG) 820 may be coupled 822 with a PUT 821 and may generate a BCG signal 825. An optical sensor 830 may be coupled 832 with a PUT 831 and may generate an optical signal 835. Coupling 832 may be to an optical element, such as a lens, a window, a light emitting diode (LED) or the like with a surface (e.g., skin) of the PUT 831 to allow emitted light 836 generated by an optical source (e.g., a light emitting diode (LED)) to enter into the PUT 831 and reflect off of a structure 834 (e.g., an artery) in PUT 831, and light 837 reflected off of structure 834 to be sensed by an optical sensor (e.g., an opto-electronic device, PIN diode, photo diode, etc.) in BCG 830. A BI sensor 840 may be coupled 842 with a PUT 841 and may generate a BI signal 845. A motion detector 850 may be coupled 852 with a PUT 851 and may generate a motion signal 855. Motion detector 850 may be included with a wearable device that includes one or more of the other sensors depicted in FIG. 8 or may be external to the wearable device as was described above in reference to FIG. 3.

In FIG. 8, BCG signal 825 may include motion signals sensed from motion sensors (e.g., an accelerometer(s)) in BCG sensor 820. Sensors 810, 820, 830, 840 and 850 may be used in one or more combinations to generate signals that are received by calibration system 880. Calibration system 880 may include a sensor selector 884 that selects one or more of the signals 815-855 received by calibration system 880 for use in a calibration process. A value on a select signal 886 (e.g., a binary value) may select which of the sensor inputs to calibration system 880 are to be used in the calibration process. Calibration system 880 may receive calibration data 881 and the calibration data 881 may be determined in part by voltage data V_{D} 882 generated by calibration system 880. Calibration system 880 may use the calibration data to generate a blood pressure (BP) signal 885.

Sensor signals selected by sensor selector 884 via values on select signal 886 may select one or more signals at the same time or at different times during the calibration process. Sensor signals selected by sensor selector 884 via values on select signal 886 may select one or more signals depending on data including but not limited to time of day (e.g., daytime, nighttime), accelerometry (e.g., from BCG 820 and/or Motion Detector 850), and temperature (e.g., ambient temperature and/or body temperature), for example. The PUT's associated with each of the depicted sensors may be on different portions of the same body, such as BI 840 coupled 842 with a wrist for PUT 841, BCG 820 coupled 822 with an ear for PUT 821, ECG 810 coupled 812 with a chest, and optical sensor 830 coupled 832 with an ear or a wrist for PUT 831, for example.

Ensembles of different sensors in FIG. 8 may be activated and their generated signals selected by sensor selector 884. As one example, pulse transit time (PTT) may be indicative of blood pressure (BP) and may be determined in part by at least two different sensor signals. Further to the example, pulse transit time (PTT) may be a speed of blood travel through an artery (e.g., the radial artery) as determined by a time from the blood being pushed from the heart to a time the blood (e.g., a pressure wave due to blood flow) arrives at the wrist. That is, pulse transit time (PTT) may be a time it takes a blood pressure pulsation to travel between two arterial sites in the body. Values of pulse transit time (PTT) may decrease due to blood velocity increases caused by an increase in blood pressure (BP). Accordingly, there may be a correlation between pulse transit time (PTT) and blood pressure (BP).

ECG sensor 810 may have its output signal 815 selected to detect a first signal indicative of the blood being pushed from the heart (e.g., a R-wave) and BI sensor 840 may have its output signal 845 selected to detect a second signal indicative of the blood pressure wave arriving at the wrist (e.g., at PUT 841). The first and second signals may be sensed from different sites on the body (e.g., at different PUT's), such as the chest for the first signal and the wrist for the second signal, for example. As another example, optical sensor 830 positioned at the wrist (e.g., a photoplethysmogram (PPG) sensor or a PulseOximeter sensor) may have its output signal 825 selected instead of the BI sensor signal 845. The first and second signals (e.g., 202 and 208 in FIG. 2) may be processed to determine the pulse arrival time (PAT) (e.g., PAT=time 208−time 202) and the pulse transit time (PTT) may be derived from the pulse arrival time (PAT) by determining the pre-ejection period (PEP) time and subtracting the PEP from the pulse arrival time (PAT) to derive the pulse transit time (PTT) (e.g., PEP≈time 204−time 202). Other methods for determining pulse transit time (PTT) may include but are not limited to determining the time interval between a peak in the R-Wave detected by ECG sensor 810 and the onset of the corresponding pressure pulse at the wrist as detected by BI sensor 840 and/or optical sensor 830 (e.g., a PPG sensor). The sensors depicted in FIG. 8 may be included in different wearable devices that are donned on different portions of the body (e.g., at different PUT's). In other examples, BCG sensor 820 may be selected, instead of ECG sensor 810, to detect the first signal.

In some examples, signals from different combinations of sensors may be selected by sensor selector 884 based on external data, such as time of day and/or accelerometry. For example, at night during periods of sleep or rest when accelerometry (e.g., as sensed by 850 and/or 820) may be reduced as compared to periods during the day where daily activities increase accelerometry, sensor selector 884 may select BCG sensor 820 instead of ECG sensor 810. Additionally, sensor selector 884 may select BI sensor 840 and/or optical sensor 830 during nighttime periods (e.g., during periods of low accelerometry). Pulse transit time (PTT) may be determined using BCG sensor 820 to detect the first signal and BI sensor 840 and/or optical sensor 830 to detect the second signal. Further to the example, during daytime periods (e.g., higher accelerometry due to motion) may select ECG sensor 810 to detect the first signal and BI sensor 840 and/or optical sensor 830 to detect the second signal. Accelerometry data may be obtained from BCG sensor 820, motion detector 850 or both.

In other examples, ECG sensor 810, BCG sensor 820 and a pulse wave sensor (e.g., BI 840 or Optical 830) may be selected by sensor selector 884. Accelerometry data may be obtained from BCG sensor 820, motion detector 850 or both.

FIG. 9 depicts examples 900 of waveforms for sensor signals. In a graph 980 of voltage amplitude vs. time, a BCG sensor 920 may generate a BCG signal 925 having a J-Wave and an ECG sensor 910 may generate an ECG signal 915 having a R-Wave. Pre-ejection period (PET) may be determined from a period of time denoted as an R-J Interval between a time for the R-Wave and a time for the J-Wave. For example, the R-J interval may be a period of time on the time axis as measured between a peak voltage of the R-Wave in signal 915 and a peak voltage of the J-Wave in signal 925. In another graph 990 of voltage amplitude vs. time, the ECG sensor 910 may generate an ECG signal 915 having a Q-Wave, and an Optical sensor 930 and/or a BI sensor 940 may generate a PPG and/or BI signal (935, 945) having a portion with a maximum slope denoted as Max Slope. Pulse arrival time may be determined by the time interval between the Q-Wave and the Max Slope. Pulse transit time (PTT) may be determined by subtracting PET from pulse arrival time (PAT) (e.g., PTT≈PAT−PET).

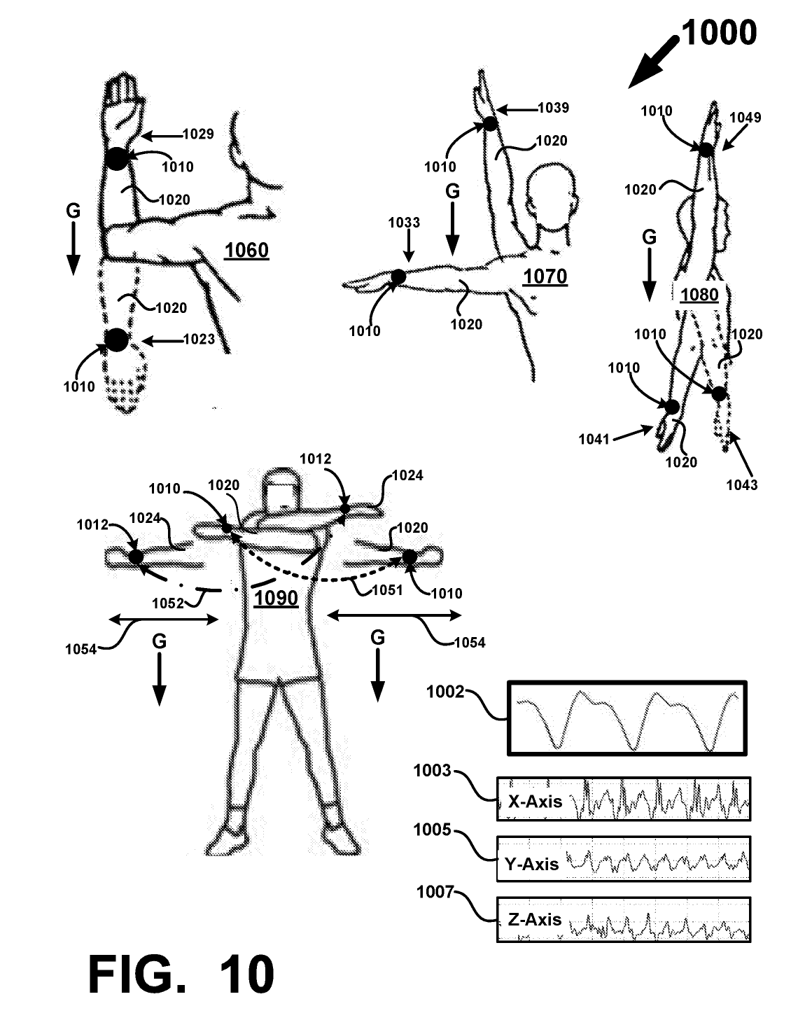

FIG. 10 depicts examples 1000 of body motion (e.g., body induced acceleration and/or angular acceleration) and sensor signals generated by the body motion that may be used for calibration of BP, for example. In FIG. 10, motion diagrams 1060-1090 depict variations in body motion of an arm (1020, 1024) on which may be mounted a wearable device (1010, 1012) that includes one or more sensors that may be used to detect accelerometry, BI, PPG, and other biometric and/or physiological signals. In motion diagram 1060, arm 1020 may be moved from a first position 1023 to a second position 1029. Motion of arm 1020 may be in opposition to gravity G when moved to from position 1023 to position 1029 and may be in cooperation with gravity G when moved from position 1029 back to position 1023. Changes in height of arm 1020 during the motion between positions 1023 and 1029 may results in accelerometry that generates motion signals and may result in changes in blood pressure (e.g., in a radial artery in arm 1020) that may be detected using BI and/or optical sensing (e.g., PPG). Other sensors, such as ECG and BCG (not shown) may also detect changes in blood pressure as manifested in their respective ECG and BCG signals.

Motion diagram 1070 depicts another example of motions of arm 1020 between positions 1033 and 1039 that may be affected blood pressure. As arm 1020 is held at position 1033, gravity G may not affect blood pressure; however, as arm 1020 is moved to position 1039, that motion may be in opposition to gravity G. Similarly, in motion diagram 1080, as arm 1020 is set into motion between positions 1041, 1043 and 1049, that motion may be in opposition to gravity G at some portions of the motion arc (e.g., proximate position 1049) and in cooperation with gravity G at other portions of the motion arc (e.g., proximate positions 1041 and 1043).

In motion diagram 1090, arm 1020 and/or arm 1024 may be swung in an arc (1051, 1052) that may be approximately perpendicular 1054 to gravity G (e.g., approximately parallel to the ground) and gravity G effects on blood pressure may be less pronounced than the gravity G effects depicted in motion diagrams 1060, 1070 and 1080, for example. Moreover, angular acceleration along a plane substantially perpendicular to gravity G (e.g., diagram 1090) may dominate acceleration effects on BP during the arc of the arm swing. Accelerometry and BI and/or PPG data may be generated by wearable device 1010, 1012, or both.

Wearable devices (1010, 1012) may generate signals 1002 indicative of changes in blood pressure due to accelerometry and/or physical exertion (e.g., from movement of arm 1020 and/or arm 1024). Wearable devices (1010, 1012) may generate motion signals 1003, 1005 and 1007 that may be used to remove motion related artifacts from signals 1002. Other sensors, such as ECG and BCG (not shown) may also detect changes in blood pressure as manifested in their respective ECG and BCG signals.

The arm movements depicted in motion diagrams 1060-1090 may be used to generate accelerometry data and biometric data associated with blood pressure (e.g., BI, ECG, BCG, PPG, PPT, pulse arrival time (PAT), PEP, etc.) and that data may be used for purposes of determining a baseline blood pressure value (e.g., P_{D} diastolic pressure or P_{S} systolic pressure) that may be specific to the individual performing the motion. The baseline data may be used for purposes of calibrating future sensor signals. The calibration procedure (e.g., the arm movements of motion diagrams 1060-1090) may be performed periodically to update and or improve accuracy in determining baseline values and/or calibrations. The calibration procedure may be performed at a specific time, such as in the morning after waking up, or at some other time, such as before going to sleep at night, for example. Wearable devices (e.g., 1010 and/or 1012) may include hardware, software or both configured for gesture recognition using signals from sensors (e.g., accelerometry and/or BI), and may process those signals to detect gestures indicative of motion (e.g., arm motion) for a calibration process, for example.

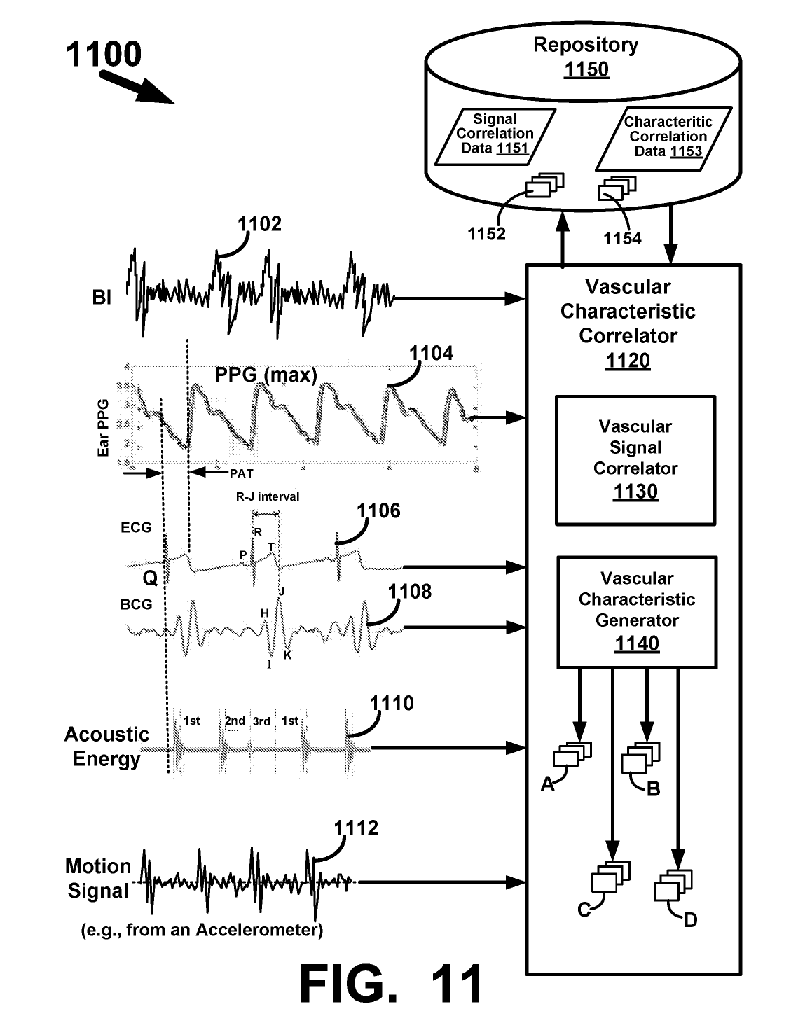

FIG. 11 depicts examples 1100 of signals generated individually or in subsets of two or more signals. FIG. 11 depicts signals for bioimpedance (BI) 1102, PPG 1104, ECG 1106, BCG 1108, acoustic energy 1110 (e.g., thumping of the heart or blood passing through an artery or a vessel, such as picked up by a piezoelectric microphone or other transducer), motion signal 1112 (e.g., an accelerometer or multi-axis accelerometer signal), or any other physiological signal embodying a physiological characteristic, such as related to blood pressure, bioimpedance, heart beat or heart rates, for example.

A repository 1150 may include signal correlation data 1151 that may be received by a vascular signal correlator 1130 to correlate physiological signals, such as those depicted in FIG. 11. Signal correlation data 1151 may include data that may be used by a vascular characteristics correlator 1120 to “align” signals (e.g., a pair of signals) as blood pulse waves passing through a vessel (e.g., the radial artery) at a certain flow rate may be correlated to one or more heart-related or vascular-related signals. For example, ECG 1106 and BCG 1108 signals may be aligned such that an R-J interval may be identified. As another example, acoustic energy signal 1110 may include a first thump and a second thump that may be related to sounds generated by the heart, which may be correlated as a signal to ECG 1106. As another example, a maximum value of PPG (e.g., at a finger) may be compared to (or may be substituted by) a BI signal 1102 (e.g., at a wrist).

Signal correlation data 1151 from repository 1150 may include signal templates 1152 of one or more of the received signals (1102, 1104, 1106, 1108, 1110, 1112) depicted in FIG. 11, whereby the signal templates 1152 may include data representing expected (e.g., empirically derived) physiological signals based on a subset of criteria, such as age, gender, ethnicity, size, height, weight, illness, infirmity, athletic prowess, and the like, for example. As such, vascular signal correlator 1130 may match a physiological signal (e.g., the BI signal 1102) derived from a sensor against a number of BI signal templates (e.g., template included in signal templates 1152) so as to normalize and/or identify portions of the physiological signals. Further, vascular signal correlator 1130 may identify portions of physiological signals, such as the ECG signal 1106 and the PPG signal 1108 to determine a pulse arrival time (PAT), for example. Note that vascular signal correlator 1130 may correlate any physiological signal to any other physiological signal to identify and extract portions of the physiological signal to generate vascular characteristics.

Vascular characteristic generator 1140 may generate data representing a subset of vascular characteristics, such as a pulse transit time (PTT) denoted as A, a vessel elasticity coefficient (E) denoted as B (e.g., a Young's Modulus of an artery, radial artery, or other blood vessel, etc.), a pulse wave velocity (PWV) denoted as C, a subset of bio impedance values (BI) denoted as D, and the like, for example. Further, vascular characteristic generator 1140 may also be configured to adapt values derived by the vascular characteristic generator 1140 (e.g., pulse transit time (PTT), vessel elasticity coefficient (E), etc.) based on characteristics correlation data 1153 stored in repository 1150. For example, sets of data 1154 representing various values of pulse transit time (PTT) may be associated with corresponding pulse transit time (PTT) correlation factor values that may be used by the vascular characteristic generator 1140 to adjust the value of pulse transit time (PTT) and deriving, for example, blood pressure (e.g., instantaneous blood pressure). Instantaneous blood pressure may be blood pressure determined in real-time while a body is in motion or at rest, for example.

Note that the retrieved physiological signals may be incorporated into the repository 1150 and may be aggregated with other similar physiological signals to generate optimized, aggregated signals from various subsets of a population.

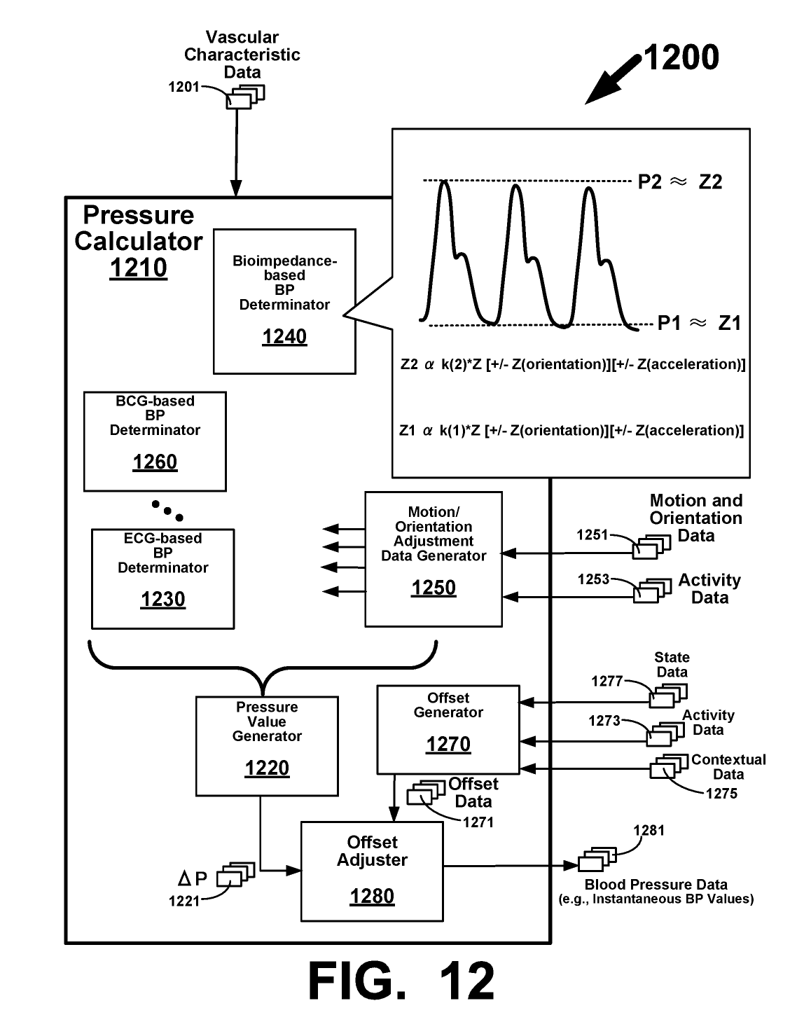

FIG. 12 depicts an example of a pressure calculator 1210 configured to determine blood pressure. Pressure calculator 1210 may be configured to determine blood pressure (BP), such as instantaneous blood pressure values or blood pressure values generated at intervals of time or aperiodically, for example. Pressure calculator 1210 may include hardware, software, and/or combination of thereof, to implement a number of blood pressure determinators, each of which may be configured to calculate and/or determine values of blood pressure in accordance with one or more aforementioned physiological signals or subsets of vascular characteristic data 1201. Pressure value generator 1210 may be configured to compare and correlate (e.g., cross-correlate) the various blood pressure values determined by the determinators so as to generate optimal (e.g., with relatively high level of accuracy) blood pressure values. For example, a pressure value generator 1220 may be configured to disregard blood pressure values generated by, for example, an BCG-based BP Determinator 1260, if the blood pressure values failed to track a threshold margin, then they may be correlated to the other BP values.

As depicted in FIG. 12, a bioimpedance-based BP determinator 1240 may generate relative values of peak (e.g., P2) and minima (e.g., P1) values of blood pressure as a function of bioimpedance (BI) values. In the example depicted, a peak pressure (e.g., a systolic pressure P_{S}) may be correlated or may be a function of a correlation factor, k(2), which may be optional, and a measured impedance value of impedance Z. In some cases, an orientation of a blood vessel relative to a source of blood (e.g., the heart) may be modeled as a contributing impedance as a function of the effects of gravity G on blood flow (e.g., see gravity G in FIG. 10). As such, the modeled impedance, Z (orientation), based on orientation may speed up blood flow when in the same direction of gravity G. For example, blood pumping through a raised arm may be affected negatively by gravity G, whereas blood flow in a lowered arm may be enhanced by gravity G. In some cases, acceleration of blood in a blood vessel relative to a point in space (e.g., a joint, the torso, or a fixed reference, such as the ground) may be modeled as a contributing impedance Z as a function of the effects of acceleration or other forces on blood flow. As such, the modeled impedance, Z (acceleration), based on forces may speed up blood flow when in the same direction of the force. For example, blood pumping through a rotating or swinging limb or body part (e.g., arm 1020 and/or arm 1024 in FIG. 10) may be affected by an angular acceleration that may affect blood pressure. Thus, modeled value of Z (acceleration) may be applied to the measured bioimpedance value to reduce or negate effects of motion.

A motion/orientation adjustment data generator 1250 may be configured to receive motion data 1251 and activity data 1253 (e.g., from one or more accelerometers, a gyroscope, or other motion sensors) and activity data 1253 may be used to generate adjustment data for adjusting the blood pressure values determined by the various determinators (e.g., the BCG-based BP Determinator 1260, the ECG-based BP Determinator 1230, and the like). For example, motion data 1251 indicating impulse forces associated with footstrikes when a user is running and/or may be identified and applied to one or more BP determinators (e.g., 1230, 1240, 1260) to reduce or negate effects of running on measured values of blood pressure. As another example, activity data 1253 representing an activity (or changes between activities) may be used to modify the determination of blood pressure values. For example if activity data 1253 suggests a user is sleeping, then resting blood pressure may be determined (which may or may not be used as a baseline). As another example, the activity data 1253 may indicate a transition from one activity to another activity, such as when a user is sleeping and awakes from sleep to change orientation by getting out of bed. The activity data 1253 may be used to modify blood pressure value determinations.

As depicted in FIG. 12, pressure value generator 1220 may be configured to generate blood pressure values 1221 (e.g., instantaneous blood pressure values) with enhanced accuracy. In some cases, pressure value generator 1220 may generate a difference in pressure (AP) that indicates relative pressure values swinging from maxima values to minima values.

In FIG. 12, an offset generator 1270 may be configured to generate offset data 1271 for consumption by an offset adjuster 1280, which may be configured to determine absolute values of blood pressure 1281 (e.g., in units of mmHg or the like). In some cases, activity data 1273 may be used to form or identify an offset. For example, if a person is sleeping for eight hours, the average or representative subset of values of the lowest blood pressure values may be to a diastolic pressure value to which an offset (e.g., from 1271) may be added to derive an absolute value of diastolic pressure (e.g., P_{D}). In other examples, the offset generator 1270 may generate an offset (e.g., from 1271) that represents an average (e.g., a moving average) of blood pressure that may be modified by a state of a user (e.g., a condition or state of health of an individual that affects or may affect blood pressure, such as whether a user is hypertensive or the like). The state of a user may be determined from state data 1277. Thus, the offset values 1271 may be relatively higher than non-hypertensive individuals. As such, the systolic and diastolic blood pressure values of the hypertensive individual may be aligned higher than non-hypertensive individuals. In at least one example, the offset generator 1270 may generate offset values 1271 based on contextual data 1275, such as whether a person has just eaten or consumed a meal (e.g., consumed a relatively large amount of glucose or other macro or micronutrients), a time of day, a level of stress, whether a person is at work or at home, whether a user is interacting socially one or more other persons, atmospheric pressure (e.g., as sensed by an altimeter), body or ambient temperature as sensed by a temperature sensor(s), or other environmental effects upon the user, and other contextual or environmental factors that may affect measurement of blood pressure values (e.g., blood pressure data 1281) or the metabolic processes that may contribute, as a response, to increases or decreases in blood pressure.

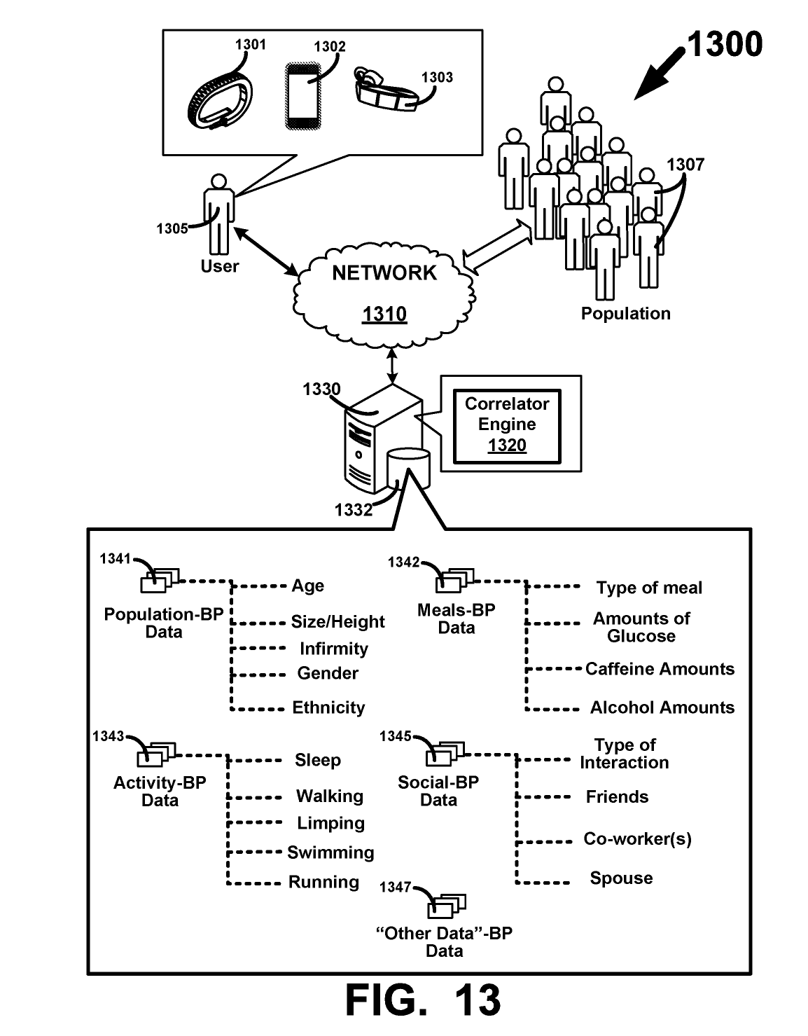

FIG. 13 depicts an example of a correlator engine 1320 configured to access a database. The correlator engine 1320 may be configured to access a database 1332 including arrangements of data that may be, for example, related to or otherwise searchable by blood pressure values. A compute engine 1330 (e.g., a server) may access data in database 1332 and may operate on data in database 1332. Correlator engine 1320 may be configured to receive data from a user 1305 (via one or more sensor-based devices such as a wearable device, a wireless device, or a mobile computing device) that describe a blood pressure and other physiological characteristics, including bioimpedance, as well as environmental and contextual characteristics of the user 1305. For example, correlator engine 1320 may receive data from one or more of devices 1301, 1302 or 1303. Correlator engine 1320 may be configured to access various data structures that may include archived or historical blood pressure values in various contexts such as a type of activity 1343, a type of meal 1342, a type of social interaction 1345, a population or sub-population 1341, or other data 1347 that may be co-related to values of blood pressure, whereby the co-related values of blood pressure may be used to derive and/or modify calculated blood pressure values to determine an instantaneous blood pressure value. In some embodiments, blood pressure values for user 1305 may be derived from a subset of blood pressure profiles based on matching demographic characteristics of the user 1305 against data from a larger population 1307 of other users. Thus, adjustments to blood pressure calculations may be based on anonymized and aggregated blood pressure values based on age, size or height of the user, gender, ethnicity, whether the user has an infirmity or illness (e.g., whether the user is hypertensive or suffers seizures), and the like.

Correlator engine 1320 may access one or more data resources that may or may not include database 1332. For example, blood pressure related data and other data may be accessed from a network 1310 (e.g., Cloud storage, the Internet, a data warehouse, RAID, a Data Farm, a server farm, a Big Data resource, NAS, or the like). Network 1310 may include data representing the population 1307 and/or subsets of data representing population 1307, for example. Sub-sets of the data representing the population 1307 may be selected to match specific physical/physiological characteristics and/or demographics of user 1305, for example. Network 1310 may include computing resources (not shown) that access data stored in network 1310 (e.g., to determine blood pressure related characteristics of user 1305).

In some examples, correlator engine 1320 may implement one or more techniques of chronicling, deriving or correlating one or more physiological characteristics are described U.S. Pat. No. 7,020,508 entitled “Apparatus For Detecting Human Physiological And Contextual Information, U.S. Pat. No. 8,641,612 entitled “Method And Apparatus For Detecting And Predicting Caloric Intake Of An Individual Utilizing Physiological And Contextual Parameters,” U.S. Pat. No. 8,369,936 entitled “Wearable Apparatus For Measuring Heart-Related Parameters and Deriving Human Status Parameters from Sensed Physiological And Contextual Parameters,” U.S. Pat. No. 8,398,546 entitled “System For Monitoring And Managing Body Weight And Other Physiological Conditions Including Iterative And Personalized Planning, Intervention And Reporting Capability,” U.S. Pat. No. 8,157,731 entitled “Method And Apparatus For Auto Journaling Of Continuous Or Discrete Body States Utilizing Physiological And/Or Contextual Parameters,” and U.S. Pat. No. 7,502,643 entitled “Method And Apparatus For Measuring Heart Related Parameters,” and the like.

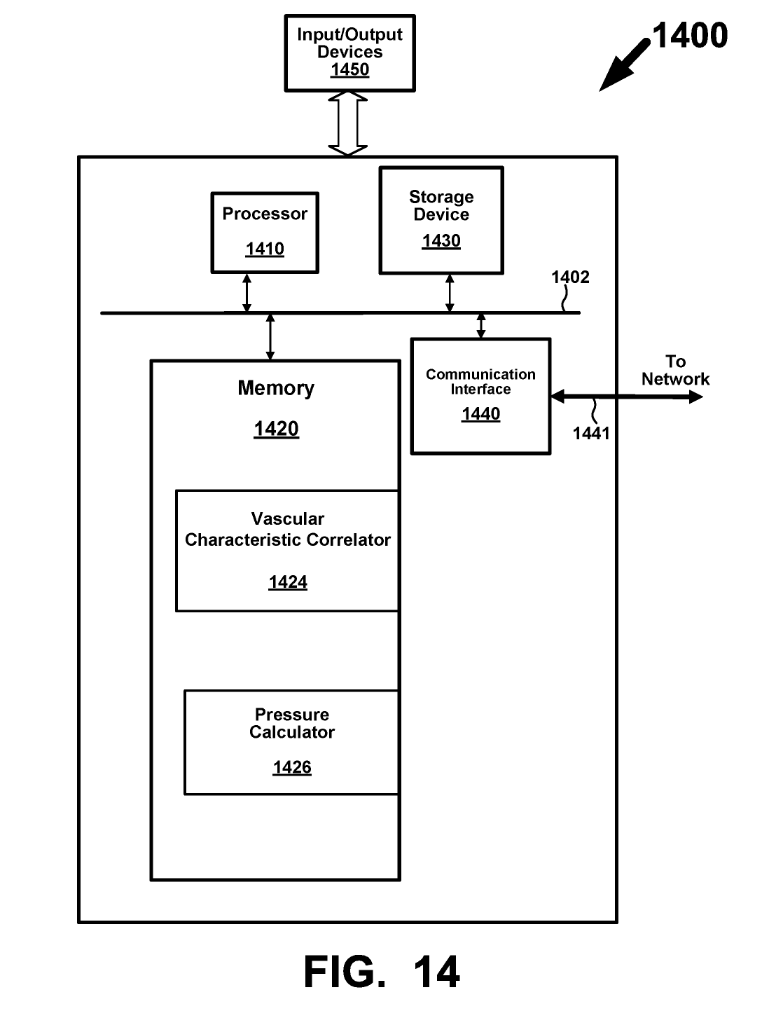

FIG. 14 depicts an example of a computing platform that may be disposed in a wearable device. In FIG. 14, an exemplary computing platform 1400 may be disposed in a wearable device (e.g., devices 1301-1303 of FIG. 13) in accordance with various embodiments. In some examples, computing platform 1400 may be used to implement computer programs, applications, methods, processes, algorithms, or other software to perform the above-described techniques. Computing platform 1400 may include a bus 1402 or other communication mechanism for communicating information, which interconnects subsystems and devices, such as processor 1410, system memory 1420 (e.g., RAM, ROM, Flash Memory, DRAM, SRAM, etc.), storage device 1430 (e.g., ROM, etc.), communication interface 1440 (e.g., an Ethernet or wireless controller, a Bluetooth controller, etc.) to facilitate communications via a port on communication link 1441 to communicate, for example, with an external computing device, including mobile computing and/or communication devices having a processor. Processor 1410 may be implemented with one or more central processing units (CPUs), such as those manufactured by Intel® Corporation, or one or more virtual processors, one or more digital signal processors (DSP's), as well as any combination of CPUs and virtual processors. Computing platform 1400 may exchange data representing inputs and outputs via input-and-output devices 1450, including, but not limited to, keyboards, mice, touch pads, a stylus, audio inputs (e.g., speech-to-text devices), user interfaces, displays, monitors, cursors, touch-sensitive displays, LCD or LED displays, and other I/O-related devices.

According to some examples, computing platform 1400 may perform specific operations by processor 1410 executing one or more sequences of one or more instructions stored in system memory 1420, and computing platform 1400 may be implemented in a client-server arrangement, peer-to-peer arrangement, or as any mobile computing device, including smart phones and the like. Such instructions or data may be read into system memory 1420 from another computer readable medium, such as storage device 1430, or network 1310 of FIG. 13, for example. In some examples, hard-wired circuitry may be used in place of or in combination with software instructions for implementation. Instructions may be embedded in software or firmware. The term “computer readable medium” refers to any tangible medium that participates in providing instructions to processor 1410 for execution. Such a medium may take many forms, including but not limited to, non-volatile media and volatile media. Non-volatile media includes, for example, Flash memory, optical or magnetic disks and the like. Volatile media includes dynamic memory (e.g., DRAM), such as system memory 1430, for example.

Common forms of computer readable media may include, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, Flash memory, any other memory chip or cartridge, or any other medium from which a computer may access data. Instructions may further be transmitted or received using a transmission medium. The term “transmission medium” may include any tangible or intangible medium that is configured to store, encode or carry instructions being configured to be executed by the machine, and may include digital or analog communications signals or other intangible medium to facilitate communication of such instructions. Transmission media may include coaxial cables, copper wire, and fiber optics, including wires that comprise bus 1402 for transmitting a computer data signal.

In some examples, execution of the sequences of instructions may be performed by computing platform 1400. According to some examples, computing platform 1400 may be coupled by communication link 1441 (e.g., a wired network, such as LAN, PSTN, or any wireless network) to any other processor or network, to perform the sequence of instructions in coordination with (or asynchronous to) one another. Computing platform 1400 may transmit and receive messages, data, and instructions, including program code (e.g., application code) through communication link 1441 and communication interface 1440. Received program code may be executed by processor 1410 as it is received, and/or stored in memory 1420 or other non-volatile storage for later execution.

In the example depicted in FIG. 14, system memory 1420 may include various modules 1424-1426 that may include executable instructions to implement functionalities described herein. In the example depicted in FIG. 14, system memory 1420 may include a vascular characteristic correlator 1424 and a pressure calculator 1426, any of which may be configured to provide one or more functions described herein.

In some embodiments, any of the above-described functions and/or structures may be implemented in and/or may be in communication (e.g., wired or wirelessly) with a mobile device, such as a mobile phone, smartphone or computing device. In some cases, a mobile device or any networked computing device (not shown) in communication with a wearable computing device may include at least some of the structures and/or functions of any of the features described herein. As depicted in one or more of the FIGS. described herein, the structures and/or functions of any of the above-described features may be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in one or more of the FIGS. described herein may represent one or more algorithms. Or, at least one of the elements may represent a portion of logic including a portion of hardware configured to provide constituent structures and/or functionalities.

For example, any of the above-described functions and/or structures may be implemented in one or more computing devices (i.e., any audio-producing device, such as desktop audio system (e.g., a Jambox® or a variant thereof)), a mobile computing device, such as a wearable device or mobile phone (whether worn or carried), that include one or more processors configured to execute one or more algorithms in memory. Thus, at least some of the elements depicted in one or more of the FIGS. described herein may represent one or more algorithms. Or, at least one of the elements may represent a portion of logic including a portion of hardware configured to provide constituent structures and/or functionalities. These may be varied and are not limited to the examples or descriptions provided.

As hardware and/or firmware, the above-described structures and techniques may be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, any of the above-described functions and/or structures may be implemented in one or more computing devices that include one or more circuits. Thus, at least one of the elements depicted in one or more of the FIGS. described herein may represent one or more components of hardware. Or, at least one of the elements may represent a portion of logic including a portion of circuit configured to provide constituent structures and/or functionalities.

According to some embodiments, the term “circuit” may refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit may include a system of electronic components and logic components (e.g., logic configured to execute instructions, such that a group of executable instructions of an algorithm, for example, and, thus, is a component of a circuit). According to some embodiments, the term “module” may refer, for example, to an algorithm or a portion thereof, and/or logic implemented in either hardware circuitry or software, or a combination thereof (i.e., a module may be implemented as a circuit). In some embodiments, algorithms and/or the memory in which the algorithms are stored are “components” of a circuit. Thus, the term “circuit” may also refer, for example, to a system of components, including algorithms or software-based modules. These may be varied and are not limited to the examples or descriptions provided.

Although the foregoing examples have been described in some detail for purposes of clarity of understanding, the above-described inventive techniques are not limited to the details provided. There are many alternative ways of implementing the above-described techniques or the present application. The disclosed examples are illustrative and not restrictive.

What is claimed is:

1. A system, comprising:
a wearable device being configured to be associated with a body;
a biometric sensor included in the wearable device, the biometric sensor being configured to generate a biometric signal indicative of biometric activity generated by a portion of the body;
a motion sensor being configured to generate a motion signal indicative of motion of the body; and
a processor being configured to:
receive the biometric signal and the motion signal,
generate data representing a difference between a first value and a second value of the biometric signal,
receive calibration data,
determine a calibration factor based on the calibration data and the data representing the difference between the first value and the second value of the biometric signal,
calculate, using the motion signal and the calibration factor, data representing a motion-related artifact in the biometric signal, and
factor the motion-related artifact out of the biometric signal to generate data representing blood pressure indicative of blood pressure in the portion of the body.

2. The system of claim 1, wherein the signal indicative of the biometric activity comprises a bioimpedance signal.

3. The system of claim 1, wherein the motion sensor is disposed in another wearable device being configured to be associated with the body.

4. The system of claim 1, wherein the motion sensor is disposed external to the body.

5. The system of claim 1, wherein the motion signal comprises accelerometry data associated with the motion of the body.

6. The system of claim 1, wherein the biometric sensor includes a plurality of electrode pairs, each electrode pair including a drive electrode and a receive electrode.

7. The system of claim 1, wherein the motion sensor comprises an accelerometer being configured to sense acceleration along at least one axis of motion.

8. The system of claim 1, wherein the calibration factor comprises the calibration data multiplied by the data representing the difference between the first value and the second value of the biometric signal.

9. The system of claim 1, wherein the biometric signal comprises a blood pressure signal.

10. The system of claim 1, wherein the biometric sensor comprises an optical sensor being configured to sense blood flow in the portion of the body.

11. The system of claim 1, wherein the processor is further configured to:
input the data representing the difference between the first value and the second value of the biometric signal to a data resource being configured to match the data representing the difference between the first value and the second value with data representing a matching value stored in the data resource, the data representing the matching value being associated with data representing an output value; and
receive the data representing the output value as the correlation data.
